(19) United States
(12) Patent Application Publication
(10) Pub. No.: US 2024/0033122 A1
(43) Pub. Date: Feb. 1, 2024

(54) POSITIONAL SLEEPING AID ASSEMBLY

(71) Applicant: KEGO Corporation, London (CA)

(72) Inventors: Kevin Gowanlock, London (CA); Nathan Pye, Woodstock (CA)

(21) Appl. No.: 17/877,074

(22) Filed: Jul. 29, 2022

(51) Int. Cl. A61F 5/56

(52) U.S. Cl. CPC A61F 5/56

(57) ABSTRACT

Provided is a garment assembly that is configured to be worn by an individual to provide lumbar support in one configuration and to prevent an individual from sleeping directly on his/her back when in bed in another configuration. The device includes a strap configured to wrap around the torso of the individual. The strap member is attachable to an elongated position control member in either a horizontal or vertical configuration relative to the torso of the individual. The elongated position control member includes an elongated flap having a thickness that is comparable to the thickness of the elongated position control member and extending from a distal end of the member. The strap member includes an attachment configuration having a pair of horizontal slots and a pair of vertical slots for receiving and supporting the flap. The flap and attachment configuration include hook and loop fasteners to allow for attachment.

### FIELD OF INVENTION

The present disclosure relates generally to a positional sleeping aid and, more particularly, a positional sleeping aid that helps to prevent and control snoring.

### BACKGROUND

Snoring affects nearly half of all adults, with many adults suffering from habitual snoring. Not only can snoring cause restlessness and exhaustion during the following day, but obstructive sleep apnea, where breathing is disrupted during sleep for short periods, may increase the risk of developing heart disease. It is estimated that 75% of people who snore have obstructive sleep apnea. Snoring can be caused, aggravated, and exasperated by sleeping on one's back. To that end, it has been found that sleeping on one's side may provide relief to snoring and may generally improve breathing.

As a result, there is a need for a comfortable, yet effective, device that prevents sleeping on one's back and encourages sleeping on one's side as a way to prevent, mitigate, or stop snoring or to generally improve breathing while sleeping. A number of prior art garments for dealing with snoring employ a pad or other member extending outwardly from the user's back, generally in the center region thereof that is supported thereon with straps or a harness. The intent of these designs is that should a person that snores rolls onto his/her back while sleeping (i.e., a supine position), a position which may cause the partial or complete closure of the individual's airway, the raised member will provide the desired degree of discomfort either to wake up the individual or to force the individual back into a prone or lateral position, i.e., a position facing downward or on his/her side. Representative assemblies that provide the above-stated function are disclosed in U.S. Pat. Nos. 6,357,444; 8,720,447; 8,015,975; 8,584,680; and U.S. Pat. Pub. No. 2012/0167895. In addition to the above patents, there are several other products on the market that disclose similar features. However, these prior art devices are not well designed to comfortably assist in addressing snoring while maintaining a person in a lateral position while sleeping.

Applicant believes that an improved garment for treating snoring is needed; one that is relatively simple in construction, and has the capability of both assisting in initially maintaining an individual in a comfortable, prone or lateral position while sleeping, while also creating a sufficient degree of discomfort when the individual moves into a supine position, to either wake the individual up or force the individual to move back into a prone or lateral position. It is further an object of the improved assembly to be adjustable to allow a user to also use the assembly to provide back related support to the user while seated or supine.

### SUMMARY

In one embodiment, provided is a garment assembly comprising an elongated position control member, such as a pillow, that includes a flap at least partially extending from a body of the elongated position control member and a strap member having an interface portion that is selectively attachable to the elongated position control member, the interface portion including a plurality of slots configured to receive a portion of the flap of the elongated position control member to support the elongated position control member in both a vertical configuration and a horizontal configuration. The interface portion may be generally continuous along the length of the strap member and made of generally common material. The plurality of slots may include a first vertical slot aligned with a second vertical slot configured to receive the flap to position the elongated position control member in the vertical configuration, and a first horizontal slot aligned with a second horizontal slot configured to receive the flap to position the elongated position control member in the horizontal configuration.

In an embodiment, the body of the elongated position control may have a length that is longer than its width, and wherein the flap includes an elongated and flat orientation wherein a length of the flap is longer than a width of the flap. The length of the flap may be longer than the length of the body. The flap may include an attachment portion that is attached to the body and an extended portion that extends from the body such that the extended portion is configured to be folded relative to the attachment portion to selectively attach the elongated position control member to the strap member. The extended portion of the flap may extend along a common axis with the body. The flap may include an outer surface and an opposite inner surface wherein the outer surface is attached to the body and the inner surface includes hook and loop fasteners to allow the flap to bias between a closed position and an open position. The plurality of slots of the interface portion may be formed by attaching a layer of material to an outer surface of the strap member. The length of the plurality of slots of the interface portion may be generally complimentary to a width of the flap. The interface portion may further include a hook and loop fastener along an outer surface that is configured to be selectively attached to the flap when assembled in the closed position along the interface portion. The elongated control member may be selectively attachable to the interface portion by inserting an extended portion of the flap within the plurality of slots and biasing an attachment portion of the flap relative to the extended portion of the flap to a closed position to engage a hook and loop fastener positioned along the attachment portion of the flap with a hook and loop fastener positioned along the extended portion of the flap.

In another embodiment, provided is a garment assembly comprising an elongated position control member including a flap at least partially extending from a body of the elongated position control member. A strap member having an interface portion that is selectively attachable to the elongated position control member, the interface portion includes a plurality of slots configured to receive a portion of the flap of the elongated position control member to support the elongated position control member in both a vertical configuration and a horizontal configuration, wherein the plurality of slots includes a first vertical slot aligned with a second vertical slot configured to receive the flap to position the elongated position control member in the vertical configuration, and a first horizontal slot aligned with a second horizontal slot configured to receive the flap to position the elongated position control member in the horizontal configuration. The interface portion may be generally continuous along the length of the strap member and made of generally common material. The flap may include an attachment portion that is attached to a body and an extended portion that extends from the body such that the extended portion is configured to be folded relative to the attachment portion to selectively attach the elongated position control member to the strap member. The extended portion of the flap may extend along a common axis with the body. The flap may include an outer surface and an opposite inner surface, wherein the outer surface is attached to the body and the inner surface includes hook and loop fasteners to allow the flap to bias between a closed position and an open position. The plurality of slots of the interface portion may be formed by attaching a layer of material to an outer surface of the strap member. The length of the plurality of slots of the interface portion may be generally complimentary to a width of the flap. The elongated control member may be selectively attachable to the interface portion by inserting an extended portion of the flap within the plurality of slots and biasing an attachment portion of the flap relative to the extended portion of the flap to a closed position to engage a hook and loop fastener positioned along the attachment portion of the flap with a hook and loop fastener positioned along the extended portion of the flap.

### BRIEF DESCRIPTION OF THE DRAWINGS

Operation of the disclosure may be better understood by reference to the following detailed description taken in connection with the following illustrations, wherein:

FIG. 1 is a perspective top view of an embodiment of a garment assembly of the present disclosure;

FIG. 2 is a top view of the garment assembly of the present disclosure, wherein an elongated position control member is aligned generally perpendicularly relative to a strap member;

FIG. 3 is a top view of the garment assembly of the present disclosure, wherein the elongated position control member is axially aligned relative to the strap member;

FIG. 4A is a rear view of the elongated position control member, with a flap member in the open position;

FIG. 4B is a side view of the elongated position control member, with the flap member in the open position;

FIG. 4C is a side view of the elongated position control member, with the flap member in a closed position;

FIG. 5A is a partial front view of a strap member in an outside open position;

FIG. 5B is a partial rear view of the strap member in the inside open position;

FIG. 6 is a partial enlarged view of a vertical attachment configuration of the strap member, with a portion of the flap member positioned through a vertical slot in the strap member;

FIG. 7A is a perspective top view of a horizontal attachment configuration of the strap member, with a portion of the flap member positioned through a horizontal slot in the strap member with the elongated position control member in the open position;

FIG. 7B is a perspective top view of a horizontal attachment configuration of the strap member, with a portion of the flap member positioned through a horizontal slot in the strap member with the elongated position control member in the closed position;

FIG. 8A is a top view of the elongated position control member attached to the strap member in the vertical attachment configuration;

FIG. 8B is a bottom view of the elongated position control member attached to the strap member in the vertical attachment configuration;

FIG. 9 is a perspective side view of the garment assembly attached to the torso of a user in the horizontal attachment configuration according to the present disclosure;

FIG. 10 is a perspective side view of the garment assembly attached to the torso of a user in the vertical attachment configuration according to the present disclosure;

FIG. 11 is a perspective view of an embodiment of the elongated position control member, with an inner support member partially exposed;

FIG. 12A is a perspective view of the inner support member separate from the elongated position control member; and

FIG. 12B is a side view of the inner support member according to the present disclosure.

### DETAILED DESCRIPTION

Reference will now be made in detail to embodiments of the present disclosure, examples of which are illustrated in the accompanying drawings. It is to be understood that other embodiments may be utilized and structural and functional changes may be made without departing from the respective scope of the disclosure. Moreover, features of the various embodiments may be combined or altered without departing from the scope of the disclosure. As such, the following description is presented by way of illustration only and should not limit in any way the various alternatives and modifications that may be made to the illustrated embodiments and still be within the spirit and scope of the disclosure.

A garment assembly 10 is disclosed and may be of any appropriate configuration and is not limited to that shown and described herein. It should similarly be understood that the garment assembly 10 may be adapted to any appropriate size and may be made of any appropriate materials and colors or with markings or indicia thereon and this disclosure is not limited in this regard.

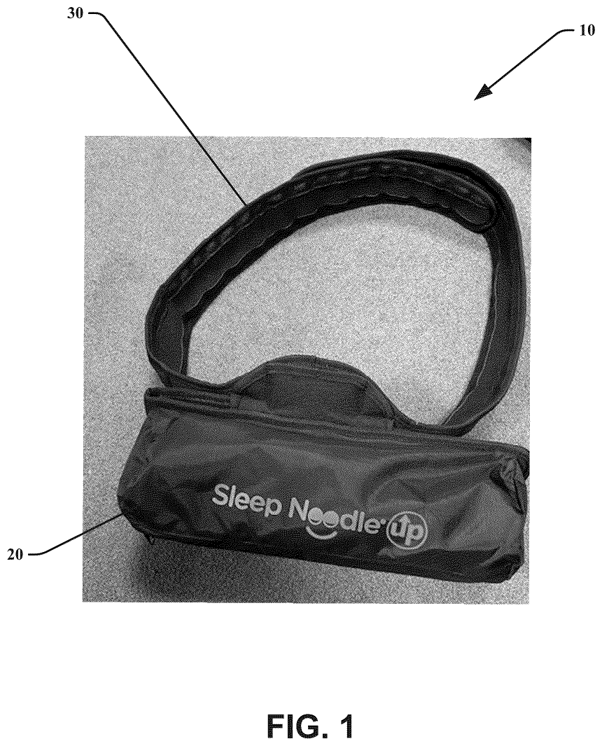

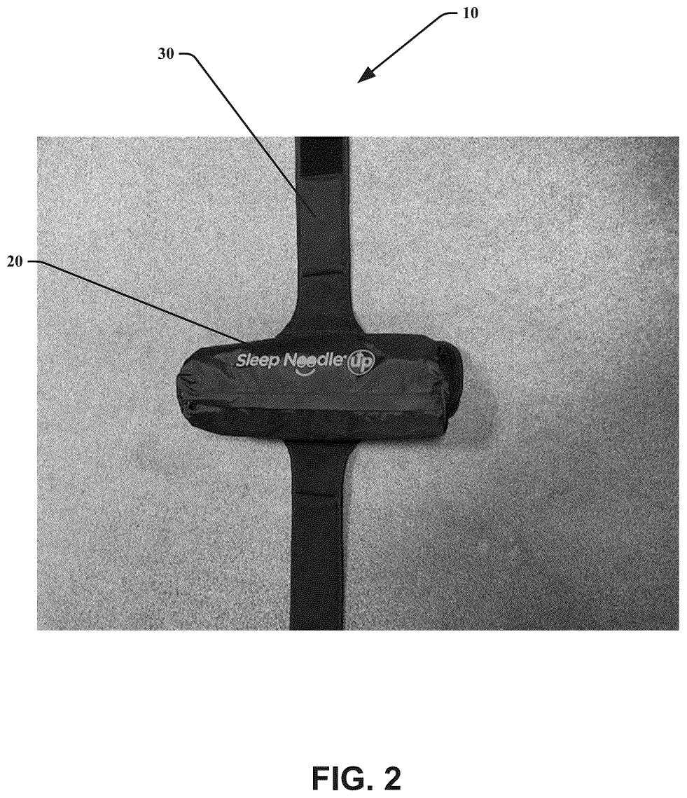

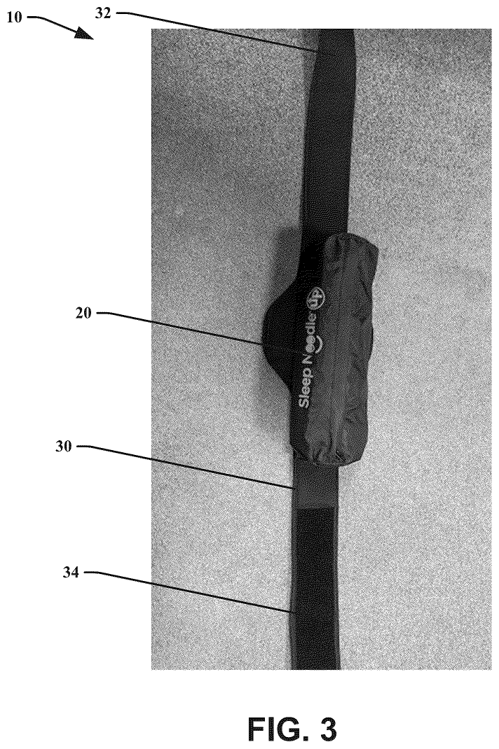

FIG. 1 is a perspective view of an embodiment of the garment assembly 10 that may include an elongated position control member 20 configured to be positioned along a back portion of the torso of a user and a strap member 30 that is configured to wrap around the torso of a user. The garment assembly 10 is configured to prevent an individual from sleeping directly on his/her back when lying in bed and the elongated position control member 20 is in the vertical configuration (FIG. 2), and to be worn by an individual to provide back support while the user is in a supine position and the elongated position control member 20 is in the horizontal configuration (FIG. 3). Notably, this assembly can be used to provide lumbar support to a user in the seated position when the elongated position control member 20 is in the horizontal configuration.

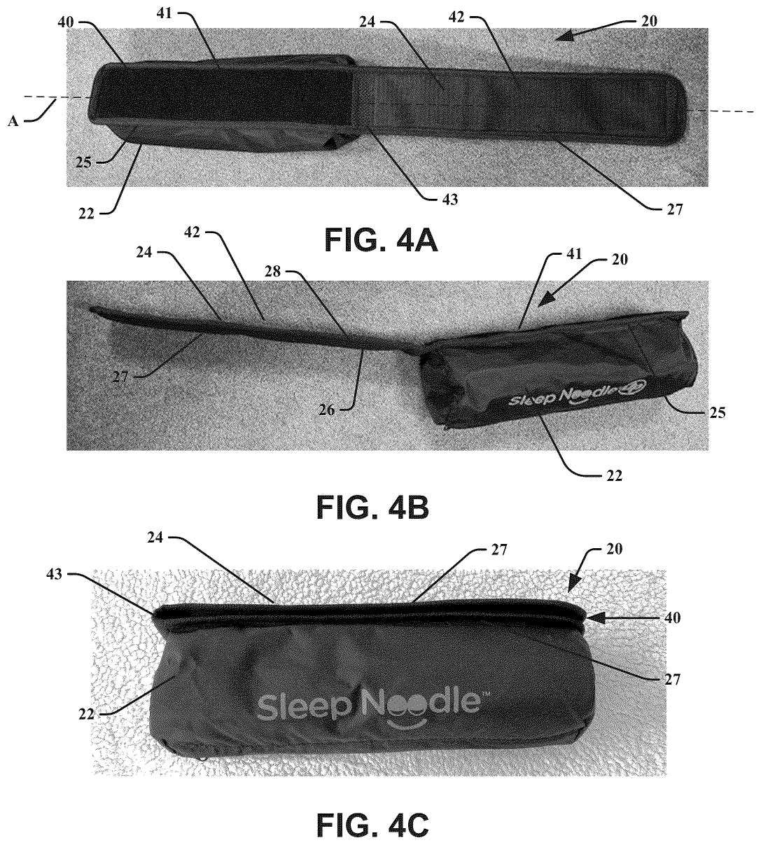

As illustrated by FIGS. 4A, 4B, and 4C, the elongated position control member 20 may include a body 22 and a flap 24, wherein the flap 24 is configured to be biased between opened and closed positions relative to the body 22. The body 22 may include an elongated and slender orientation wherein the length is longer than the width. The flap 24 may include an elongated and flat orientation wherein the length is longer than the width. In one embodiment, the length of the flap 24 may be longer than the length of the body 22. The length of the flap 24 may be at or about twice the length of the body 22. The flap 24 may include an attachment portion 25 that is attached to the body 22 and an extended portion 27 that extends from the body 22 such that the extended portion 27 can be folded relative to the attachment portion 25 to be selectively attached to the strap member 30. In an embodiment, the length of the attachment portion 25 of the flap 24 may be attached along the length of the body 22 such that the extended portion 27 extends from the attachment portion 25 along a common axis A with the body 22.

The flap 24 may include an outer surface 26 and an opposite inner surface 28. The outer surface 26 may be attached to the body 22 along the attachment portion 25 and the inner surface 28 may include hook and loop fasteners 40 to allow the extended portion 27 to be biased into the closed position against the attachment portion 25. The loop portion 41 of the hook and loop fasteners 40 may be placed along the attachment portion 25 on the inner surface 28, while the hook portion 42 of the hook and loop fasteners 40 may be placed along the extended portion 27 of the inner surface 28. The extended portion 27 may be biased relative to the attachment portion 25 along a hinge 43. This allows a substantial portion of the extended portion 27 of the flap 24 to be attached to a substantial portion of the attachment portion 25 along the inner surface 28 as illustrated by FIG. 4C. This will also allow for the elongated position control member 20 to be placed in adjustable relation to the strap member 30 as disclosed further herein.

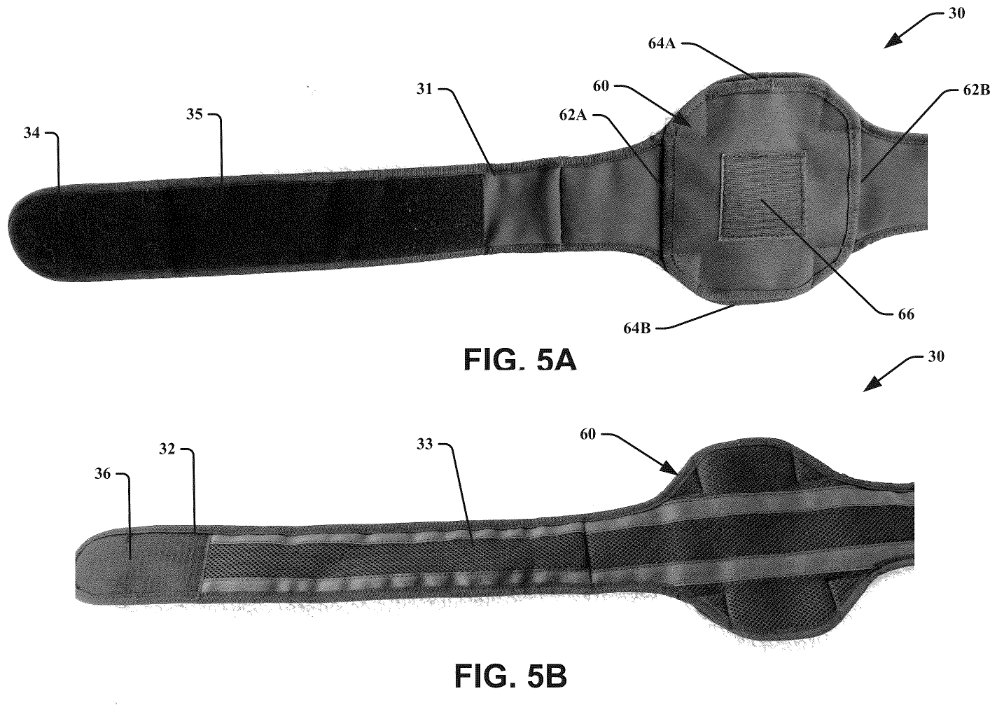

FIGS. 5A and 5B illustrate portions of the strap member 30. FIG. 5A illustrates an outer surface 31 of the strap member 30 while FIG. 5B illustrates an inner surface 33 of the strap member 30 wherein the inner surface is configured to be in contact with the torso of the user. The strap member 30 may be elongated and extend from a first end portion 32 to an opposite second end portion 34 (See FIG. 3). The strap member 30 may also include a fastener such as hook and loop fasteners that are configured to allow opposing surfaces of the first end portion 32 to be adjustably attached to the second end portion 34. However, this disclosure contemplates that any type of fastener may be utilized including, buttons, buckles, clips, snaps, etc. and this disclosure is not limited herein. The strap member 30 allows a user to comfortably wrap the strap member 30 and position member 20 around his/her torso in an adjustable and comfortable manner. As illustrated by FIGS. 5A and 5B, loop portions 35 of the hook and loop fasteners may extend along a length of the outer surface 31 of the second end 34 while hook portions 36 may extend along a length of the inner surface 33 of the first end 32. The fastener portions may be interchangeable along each surface but may be generally configured to allow the strap member 30 to be wrapped around various sized torsos for comfortable attachment thereto.

The strap member 30 may include an interface portion 60 that is configured to receive and support the elongated position control member 20 relative to the strap member 30. The interface portion 60 may be generally continuous along the length of the strap member 30 and be made of generally common material to the first and second ends 32, 34. The interface portion 60 may include a plurality of slots 62, 64 configured to receive a portion of the flap 24 of the elongated position control member 20 therein to support the elongated position control member 20 in both an adjustable horizontal position and an adjustable vertical position. The slots may be formed by a layer of material attached to the outer surface of the interface portion 60 wherein the length of the slots 62A, 62B, 64A, 64B are generally complimentary to the width of the extended portion 27 of the flap 24. Further, the interface portion 60 may include a hook and loop fastener 66 along the outer surface configured for attachment with the hook and loop fastener along the attachment portion 25 of the flap 24 when assembled in the closed position along the interface portion 60.

First and second horizontal slots 62A, 62B are aligned relative to one another along a common axis and configured to receive the flap 24 to allow the elongated position control member 20 to be arranged in the horizontal position relative to the strap member 30. Similarly, first and second vertical slots 64A, 64B are configured and aligned to receive the flap 24 to allow the elongated position control member 20 to be arranged in the vertical position relative to the strap member 30.

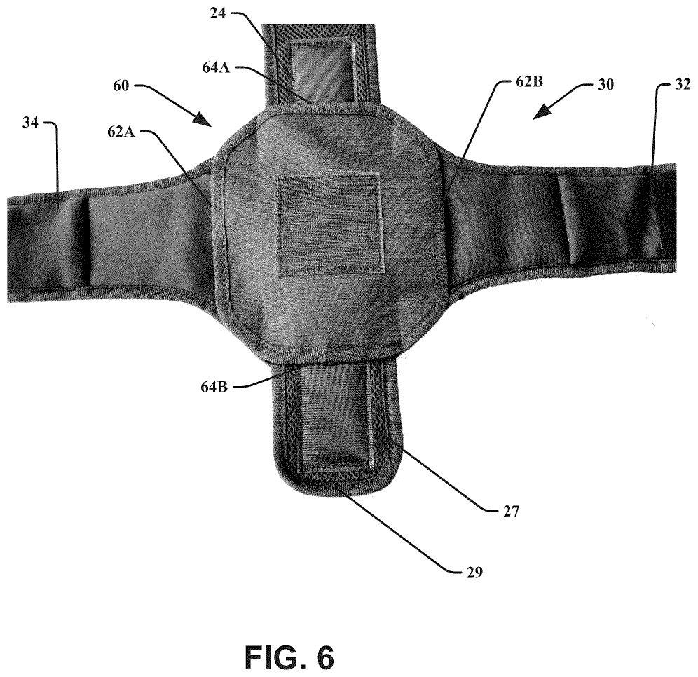

FIG. 6 illustrates a portion of the flap 24 of the elongated position control member 20 inserted within the interface portion 60 of the strap member 30 to be attached in the vertical position. Here, a tip 29 of the extended portion 27 is inserted within the first vertical slot 64A and extends through to the aligned second vertical slot 64B. The body 22 of the elongated control member 20 may then be biased relative to the flap 24 to the closed position to engage the hook and loop fastener of the attachment portion 25 of the flap member with the hook and loop fastener of the extended portion 27 of the flap member 24 to configure the garment assembly in the vertical configuration as illustrated by FIG. 2. Notably, the hook and loop fastener 66 on the interface portion 60 may be selectively attached to the hook and loop fastener 40 along the attachment portion 25 of the elongated position control member 20.

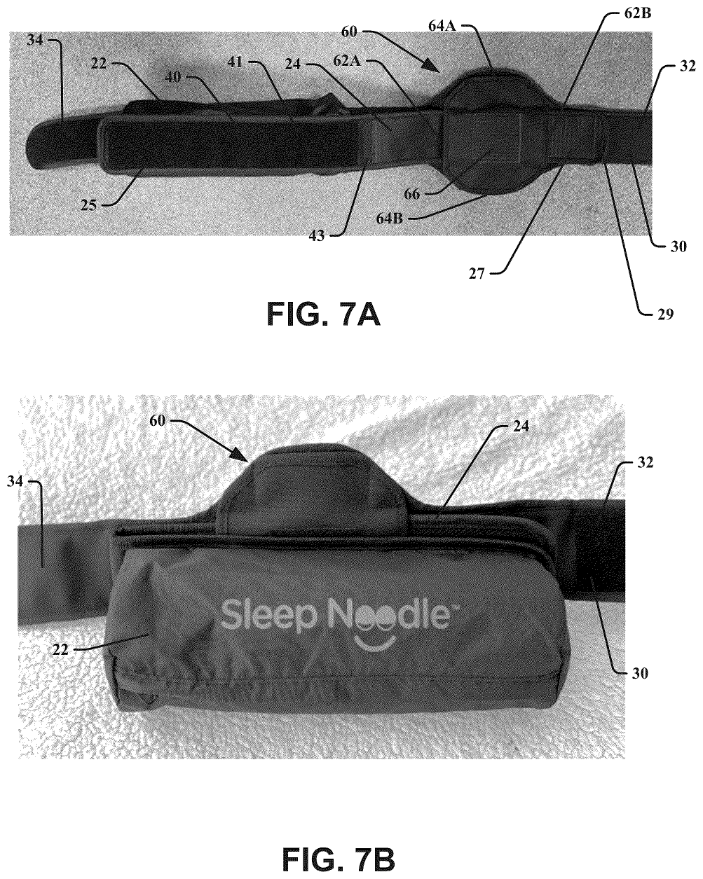

FIG. 7A illustrates a portion of the flap 24 of the elongated position control member 20 inserted within the interface portion 60 of the strap member 30 to be attached in the horizontal position. Here, the tip 29 of the extended portion 27 is inserted within the first horizontal slot 62A and extends through to the aligned second horizontal slot 62B. The body 22 of the elongated control member 20 may then be biased relative to the flap 24 to the closed position to engage the hook and loop fastener of the attachment portion 25 of the flap member 24 with the hook and loop fastener of the extended portion 27 of the flap member 24 to configure the garment assembly in the horizontal configuration as illustrated by FIG. 7B. Notably, the hook and loop fastener 66 on the interface portion 60 may be selectively attached to the hook and loop fastener 40 along the attachment portion 25 of the elongated position control member 20.

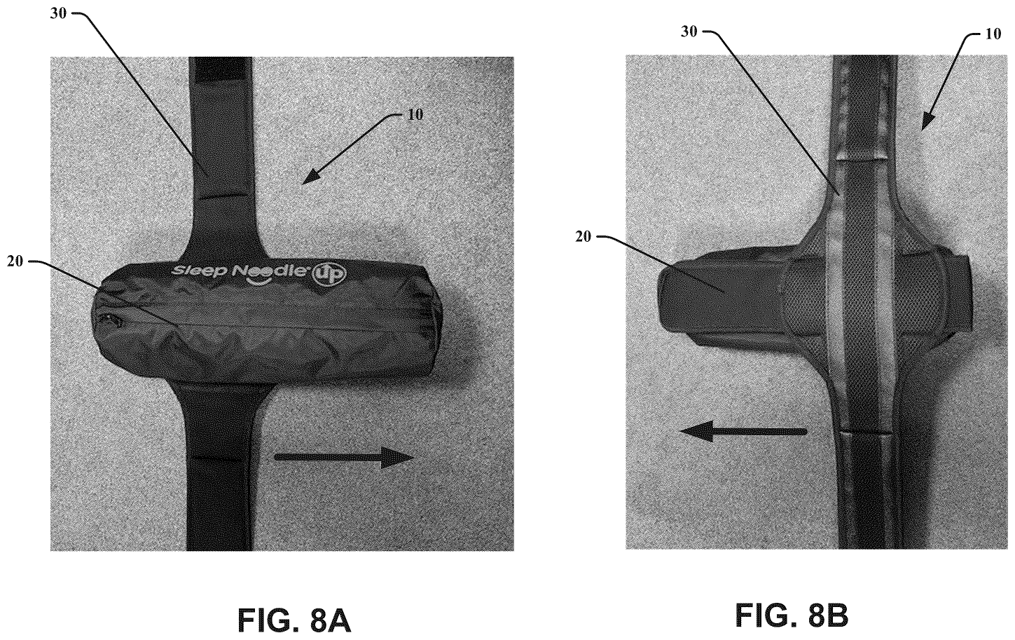

In one embodiment as illustrated in at least FIGS. 7A and 7B, the flap 24 may have a thickness dimension that is approximately equal to the thickness dimension of the first and second ends 32, 34 of the strap member 30. Further, the strap 27 may be adjustable relative to the interface portion, such that the extended portion 27 of the flap member may be translated relative to its placement within the slots 62A, 62B, 64A, 64B, allowing the member 20 to raise or lower towards a left or right side as positioned on the back of a user. FIG. 8A illustrates a top view of the elongated position control member 20 in the vertical attachment configuration in an adjusted position relative to the strap member 30 as illustrated by the arrow. Similarly, FIG. 8B illustrates a bottom view of the elongated position control member 20 in the vertical attachment configuration in an adjusted position relative to the strap member 30 as illustrated by the arrow.

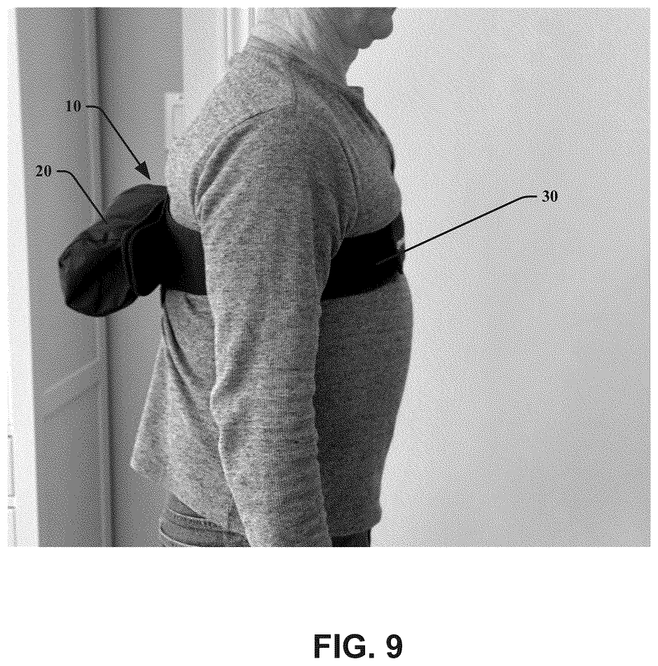

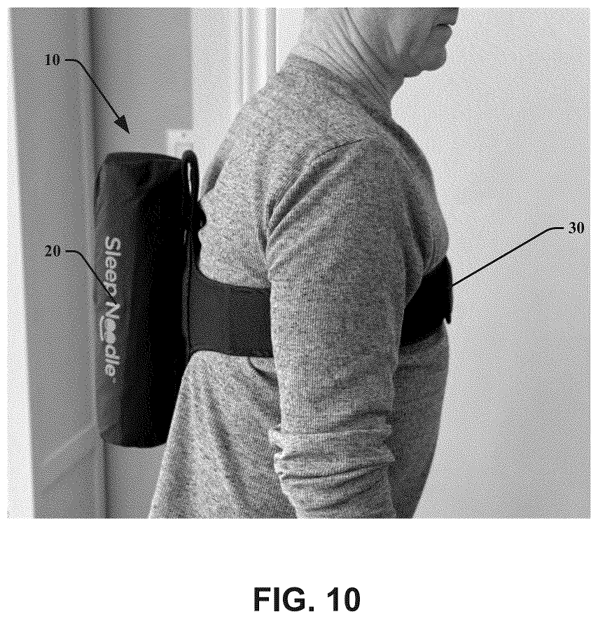

FIG. 9 is a perspective side view of the garment assembly 10 attached to the torso of a user in the horizontal attachment configuration, and FIG. 10 is a perspective side view of the garment assembly 10 attached to the torso of a user in the vertical attachment configuration.

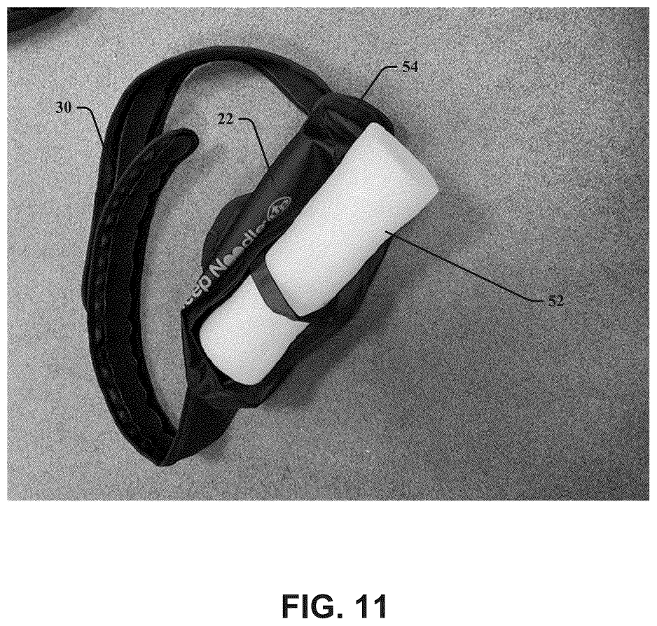

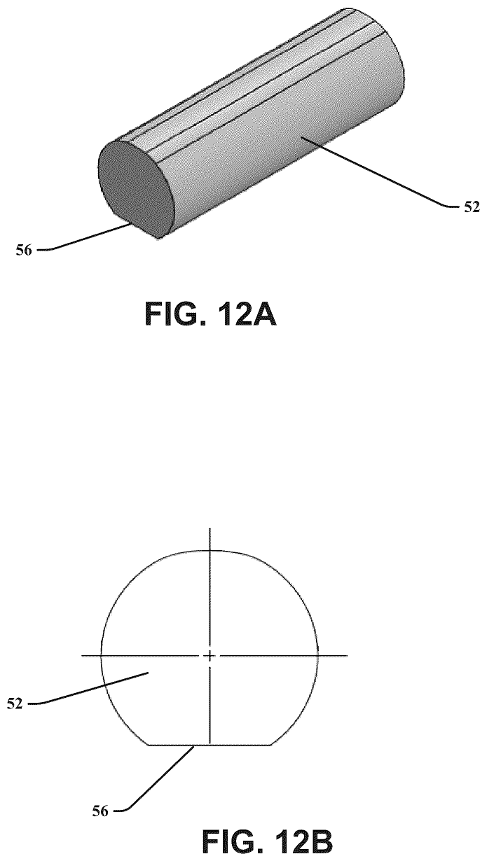

FIG. 11 illustrates that the body 22 may include in insert 52 surrounded by a garment material 54 that is configured to be comfortable to a user and allows for a snug fit to users of various sized torsos. The insert 52 may include a generally elongated and cylindrical shape. Notably, the insert 52 may have various shapes and sizes but may be similar to the appearance of a foam pool toy that includes a hole through the middle. As illustrated by FIGS. 12A and 12B, the outer surface of the insert 52 may also include a flat portion 56 to allow the insert to align with the attachment portion of the flap member 24 as it is inserted into the garment material 54. In an embodiment, the flat portion 56 of the insert 52 can be aligned to be positioned against the back of a user when worn. The garment material 54 may be formed to define a cavity and have an opening to allow the insert 52 to be removed from the cavity or contained therein. A zipper or other type of garment fastening device, such as straps, snaps, or buttons, may be used to selectively open or close the opening of the garment material 54. The insert 52 may be removed and replaced from the garment material 54 to be washed separately from the insert 52.

Stated another way, the device includes a strap configured to wrap around the torso of the individual. The strap member is attachable to an elongated position control member (i.e., the pillow) in either a horizontal or vertical configuration relative to the torso of the individual. The elongated position control member includes an elongated flap having a thickness that is comparable to the thickness of the elongated position control member and extending from a distal end of the member. The strap member includes an attachment configuration having a pair of horizontal slots and a pair of vertical slots for receiving and supporting the flap. The flap and attachment configuration include hook and loop fasteners to allow for attachment.

Although the embodiments of the present disclosure have been illustrated in the accompanying drawings and described in the foregoing detailed description, it is to be understood that the present invention is not to be limited to just the embodiments disclosed, but that the disclosure described herein is capable of numerous rearrangements, modifications and substitutions without departing from the scope of the claims hereafter. The features of each embodiment described and shown herein may be combined with the features of the other embodiments described herein. The claims as follows are intended to include all modifications and alterations insofar as they come within the scope of the claims or the equivalent thereof.

Having thus described the invention, we claim:

1. A garment assembly comprising:
an elongated position control member including a flap at least partially extending from a body of the elongated position control member; and
a strap member having an interface portion that is selectively attachable to the elongated position control member, the interface portion including a plurality of slots configured to receive a portion of the flap of the elongated position control member to support the elongated position control member in both a vertical configuration and a horizontal configuration.

2. The garment assembly of claim 1, wherein the interface portion is generally continuous along the length of the strap member and made of generally common material.

3. The garment assembly of claim 1, wherein the plurality of slots includes a first vertical slot aligned with a second vertical slot configured to receive the flap to position the elongated position control member in the vertical configuration, and a first horizontal slot aligned with a second horizontal slot configured to receive the flap to position the elongated position control member in the horizontal configuration.

4. The garment assembly of claim 1, wherein the elongated position control member includes a body that has a length that is longer than the width, and wherein the flap includes an elongated and flat orientation wherein a length of the flap is longer than a width of the flap.

5. The garment assembly of claim 4, wherein the length of the flap is longer than the length of the body.

6. The garment assembly of claim 1, wherein the flap includes an attachment portion that is attached to a body and an extended portion that extends from the body such that the extended portion is configured to be folded relative to the attachment portion to selectively attach the elongated position control member to the strap member.

7. The garment assembly of claim 6, wherein the extended portion of the flap extends along a common axis with the body.

8. The garment assembly of claim 1, wherein the flap includes an outer surface and an opposite inner surface, wherein the outer surface is attached to the body and the inner surface includes hook and loop fasteners to allow the flap to bias between a closed position and an open position.

9. The garment assembly of claim 1, wherein the plurality of slots of the interface portion are formed by attaching a layer of material to an outer surface of the strap member.

10. The garment assembly of claim 1, wherein the length of the plurality of slots of the interface portion are generally complimentary to a width of the flap.

11. The garment assembly of claim 1, wherein the interface portion further includes a hook and loop fastener along an outer surface that is configured to be selectively attached to the flap when assembled in the closed position along the interface portion.

12. The garment assembly of claim 1, wherein the elongated control member is selectively attachable to the interface portion by inserting an extended portion of the flap within the plurality of slots and biasing an attachment portion of the flap relative to the extended portion of the flap to a closed position to engage a hook and loop fastener positioned along the attachment portion of the flap with a hook and loop fastener positioned along the extended portion of the flap.

13. A garment assembly comprising:
an elongated position control member including a flap at least partially extending from a body of the elongated position control member;
a strap member having an interface portion that is selectively attachable to the elongated position control member, the interface portion including a plurality of slots configured to receive a portion of the flap of the elongated position control member to support the elongated position control member in both a vertical configuration and a horizontal configuration; and
wherein the plurality of slots includes a first vertical slot aligned with a second vertical slot configured to receive the flap to position the elongated position control member in the vertical configuration, and a first horizontal slot aligned with a second horizontal slot configured to receive the flap to position the elongated position control member in the horizontal configuration.

14. The garment assembly of claim 13, wherein the interface portion is generally continuous along the length of the strap member and made of generally common material.

15. The garment assembly of claim 13, wherein the flap includes an attachment portion that is attached to a body and an extended portion that extends from the body such that the extended portion is configured to be folded relative to the attachment portion to be selectively attach the elongated position control member to the strap member.

16. The garment assembly of claim 15, wherein the extended portion of the flap extends along a common axis with the body.

17. The garment assembly of claim 13, wherein the flap includes an outer surface and an opposite inner surface, wherein the outer surface is attached to the body and the inner surface includes hook and loop fasteners to allow the flap to bias between a closed position and an open position.

18. The garment assembly of claim 13, wherein the plurality of slots of the interface portion are formed by attaching a layer of material to an outer surface of the strap member.

19. The garment assembly of claim 13, wherein the length of the plurality of slots of the interface portion are generally complimentary to a width of the flap.

20. The garment assembly of claim 13, wherein the elongated control member is selectively attachable to the interface portion by inserting an extended portion of the flap within the plurality of slots and biasing an attachment portion of the flap relative to the extended portion of the flap to a closed position to engage a hook and loop fastener positioned along the attachment portion of the flap with a hook and loop fastener positioned along the extended portion of the flap.
